(19) United States
(12) Patent Application Publication
(10) Pub. No.: US 2005/0119328 A1
(43) Pub. Date: Jun. 2, 2005

(54) Novel crysalline forms of tegaserod maleate

(75) Inventors: Reddy Bandi Parthasaradhi, Hyderabad (IN); Reddy Kura Rathnakar, Hyderabad (IN); Reddy Rapolu Raji, Hyderabad (IN); Reddy Dasari Muralidhara, Hyderabad (IN); Reddy Itiyala Srinivas, Andhrapradesh (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad (IN)

(21) Appl. No.: 10/508,905

(22) PCT Filed: Mar. 25, 2003
(86) PCT No.: PCT/IN03/00076

(51) Int. Cl. A61K 31/405; C07D 209/14

(52) U.S. Cl. 514/419; 548/505

(57) ABSTRACT

The present invention relates to novel crystalline forms of tegaserod maleate, to processes for their preparation and to pharmaceutical compositions containing them.

### FIELD OF THE INVENTION

The present invention relates to novel crystalline forms of tegaserod maleate, to processes for their preparation and to pharmaceutical compositions containing them.

### BACKGROUND OF THE INVENTION

EP Patent No. 0 442,378 describes, along with other compounds, the compound (1) 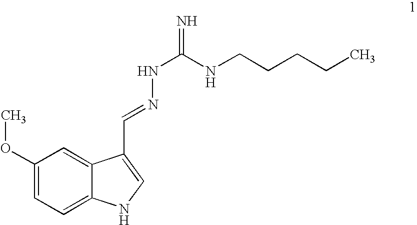
or 2-[(5-Methoxy-1H-indol-3-yl)methylene]-N-pentylhydrazinecarboximidamide, which has the generic name tegaserod and forms maleic acid salt (tagaserod maleate). Tegaserod and related compounds are serotonin 5HT₄-receptor partial agonist and useful in the treatment of irritable bowel syndrome and other utilities as described in EP Patent No. 0 442,378.

Crystalline forms of tegaserod maleate have not been reported in the literature and also, the preparation of tegaserod maleate has not been described. So, there is a need for stable polymorphs of tegaserod maleate for better pharmaceutical preparations.

It has now been discovered that tegaserod maleate can be prepared in four different crystalline forms.

Thus the object of the present invention is to provide stable novel crystalline forms of tegaserod maleate, processes for preparing these forms and pharmaceutical compositions containing them.

### DETAILED DESCRIPTION OF THE INVENTION

In accordance with the present invention, there is provided a novel crystalline form of tegaserod maleate, designated as Form I, characterized by an x-ray powder diffraction pattern having peaks expressed as 2θ at about 5.3, 5.9, 6.4, 10.7, 16.1 and 26.8 degrees. FIG. 1 shows typical Form I x-ray powder diffraction pattern.

In accordance with the present invention, a process is provided for preparation of tegaserod maleate Form I. In this process, maleic acid is added to a solution of tegaserod free base in acetone and tegaserod maleate Form I is isolated from the mixture. Tegaserod maleate Form I may be isolated by usual techniques like cooling, partial removal of the solvent from the solution, adding an anti-solvent.

In accordance with the present invention, an alternative process is provided for preparation of tegaserod maleate Form I. According to this process, tegaserod maleate is mixed with acetone and collecting tegaserod maleate Form I from the mixture by filtration. In this process any of the crystalline forms of tegaserod maleate may be used.

In accordance with the present invention, there is provided a novel crystalline form of tegaserod maleate, designated as Form II, characterized by an x-ray powder diffraction pattern having peaks expressed as 2θ at about 5.3, 6.4, 6.9, 7.8, 8.7, 10.2, 10.8, 15.5, 16.8, 17.0, 19.5, 21.2, 21.7, 22.7 and 25.2 degrees. FIG. 2 shows typical Form II x-ray powder diffraction pattern.

In accordance with the present invention, a process is provided for preparation of tegaserod maleate Form II. In this process, tegaserod maleate is dissolved in methanol and tegaserod maleate Form II is precipitated from the solution by adding acetonitrile. In this process any of the crystalline forms of tegaserod maleate may be used may be used to prepare the solution in methanol.

In accordance with the present invention, there is provided a novel crystalline form of tegaserod maleate, designated as Form III, characterized by an x-ray powder diffraction pattern having peaks expressed as 2θ at about 7.0, 7.9, 8.7, 10.2, 15.6, 15.9, 17.0, 19.5, 25.3 and 27.1 degrees. FIG. 3 shows typical Form III x-ray powder diffraction pattern.

In accordance with the present invention, a process is provided for preparation of tegaserod maleate Form III. In this process, maleic acid is added to a solution of tegaserod free base in methanol and the contents are maintained for about 30 minutes at about 20° C. to 25° C. and then the crystals are collected by filtration.

In accordance with the present invention, another process is provided for preparation of tegaserod maleate Form III. According to this process, tegaserod maleate is dissolved in methanol and the solution is maintained for about 30 minutes at about 20° C. to 25° C. and then tegaserod maleate Form III crystals are collected by filtration.

In accordance with the present invention, there is provided a novel crystalline form of tegaserod maleate, designated as Form IV, characterized by an x-ray powder diffraction pattern having peaks expressed as 2θ at about 6.9, 8.0, 10.3, 16.5, 19.6, 20.4, 20.9, 22.0, 23.2, 25.4, 28.0 and 28.7 degrees. FIG. 4 shows typical Form IV x-ray powder diffraction pattern.

In accordance with the present invention, a process is provided for preparation of tegaserod maleate Form IV. In this process, maleic acid is added to a solution of tegaserod free base in methanol and tegaserod maleate Form IV is precipitated by adding methylene dichloride or isopropyl alcohol.

Tegaserod free base used in the above processes may be obtained by the procedures described in EP Patent No. 0 442,378.

In accordance with the present invention, there is provided a pharmaceutical composition comprising crystalline form of tegaserod maleate and a pharmaceutically acceptable carrier.

### BRIEF DESCRIPTION OF THE DRAWINGS

FIG. 1 is a x-ray powder diffraction pattern of tegaserod maleate Form I.

FIG. 2 is a x-ray powder diffraction pattern of tegaserod maleate Form II.

FIG. 3 is a x-ray powder diffraction pattern of tegaserod maleate Form III.

FIG. 4 is a x-ray powder diffraction pattern of tegaserod maleate Form IV.

x-Ray powder diffraction spectrum was measured on a Siemens D5000 x-ray powder diffractometer having a copper-Kα radiation.

The following examples further illustrate the invention.

### EXAMPLE 1

Tegaserod free base (10 gm) is dissolved in acetone (100 ml). Maleic acid (4 gm) is added to the solution and the contents are maintained for 1 hour at 25° C. The separated solid is filtered to give 12.5 gm of tegaserod maleate Form I.

### EXAMPLE 2

Tegaserod maleate Form II (5 gm) and acetone (70 ml) are mixed and refluxed for 1 hour and cooled to 25° C. and filtered to give 4.8 gm of tegaserod maleate Form I.

### EXAMPLE 3

Tegaserod maleate Form I (10 gm) is dissolved in methanol (100 ml). Acetonitrile (150 ml) is added to the solution and the contents are heated to reflux. The contents are then cooled to 25° C. and maintained for 30 minutes. The separated crystals are collected by filtration to give 9 gm of tegaserod maleate Form II.

### EXAMPLE 4

Tegaserod free base (10 gm) is dissolved in methanol (100 ml) and maleic acid (4 gm) is added to the solution. Then the contents are maintained for 30 minutes at 25° C. Then the separated solid is filtered to give 13 gm of tegaserod maleate Form III.

### EXAMPLE 5

Tegaserod maleate (5 gm) is dissolved in methanol (50 ml) and the solution is maintained at 25° C. for 30 minutes. The separated crystals are collected by filtration to give 4.8 gm of tegaserod maleate Form III.

### EXAMPLE 6

Tegaserod free base (10 gm) is dissolved in methanol (50 ml), maleic acid (4 gm) is added and the contents are refluxed for 30 minutes and then the resulting solution is cooled to 25° C. Methylene dichloride (200 ml) is added and the contents are maintained for 30 minutes at 25° C. The separated solid is collected by filtration to give 13 gm of tegaserod maleate Form IV.

### EXAMPLE 7

Maleic acid (4 gm) is added to a solution of tegaserod free base (10 gm) in methanol (50 ml). The contents are maintained for 30 minutes at 25° C. and isopropyl alcohol (150 ml) is mixed and contents are maintained for 30 minutes at 25° C. The separated solid is collected by filtration to give 12.5 gm of tegaserod maleate Form IV.

1. A crystalline tegaserod maleate Form I, characterized by an x-ray powder diffraction pattern having peaks expressed as 2θ at about 5.3, 5.9, 6.4, 10.7, 16.1 and 26.8 degrees.

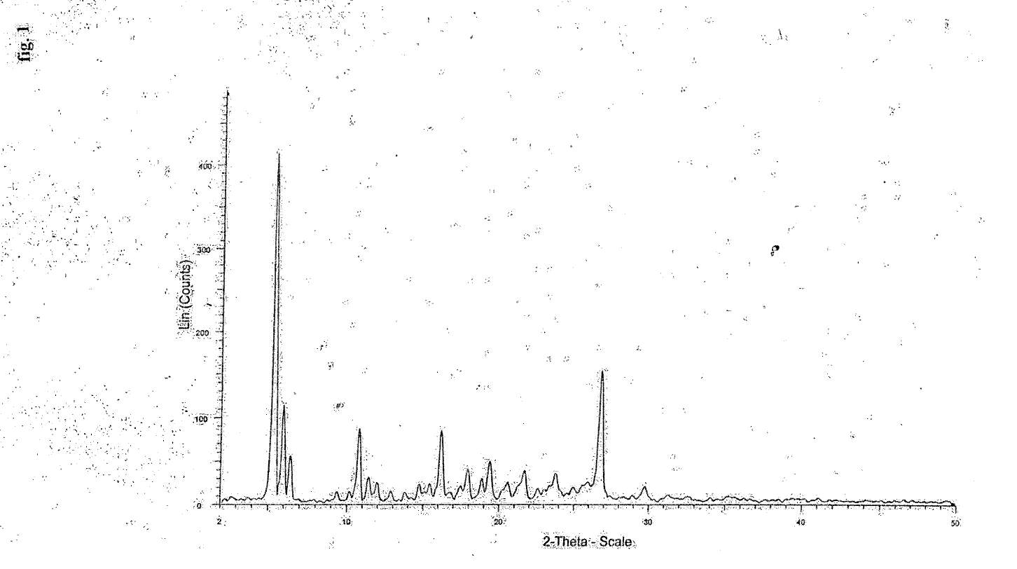

2. A crystalline tegaserod maleate Form I as defined in claim 1, further characterized by an x-ray powder diffraction pattern as shown in FIG. 1.

3. A process for preparing tegaserod maleate Form I as defined in claim 1, which comprises:
a) adding maleic acid to a solution of tegaserod free base in acetone; and
b) Isolating tegaserod maleate Form I.

4. A process of preparing tegaserod maleate Form I as defined in claim 1, which comprises mixing tegaserod maleate and acetone and collecting tegaserod maleate Form I by filtration.

5. A crystalline tegaserod maleate Form II, characterized by an x-ray powder diffraction pattern having peaks expressed as 2θ at about 5.3, 6.4, 6.9, 7.8, 8.7, 10.2, 10.8, 15.5, 16.8, 17.0, 19.5, 21.2, 21.7, 22.7 and 25.2 degrees.

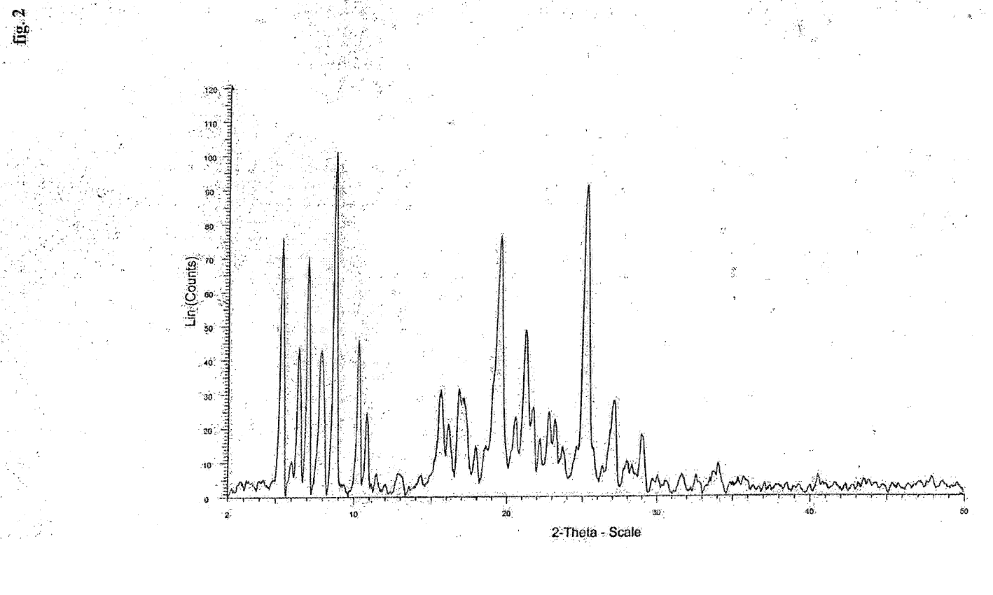

6. A crystalline tegaserod maleate Form II as defined in claim 5, further characterized by an x-ray powder diffraction pattern as shown in FIG. 2.

7. A process for preparing tegaserod maleate Form II as defined in claim 5, which comprises:
a) dissolving tegaserod maleate in methanol; and
b) precipitating tegaserod maleate Form II from the solution by mixing with acetonitrile;

8. A crystalline tegaserod maleate Form III, characterized by an x-ray powder diffraction pattern having peaks expressed as 2θ at about 7.0, 7.9, 8.7, 10.2, 15.6, 15.9, 17.0, 19.5, 25.3 and 27.1 degrees.

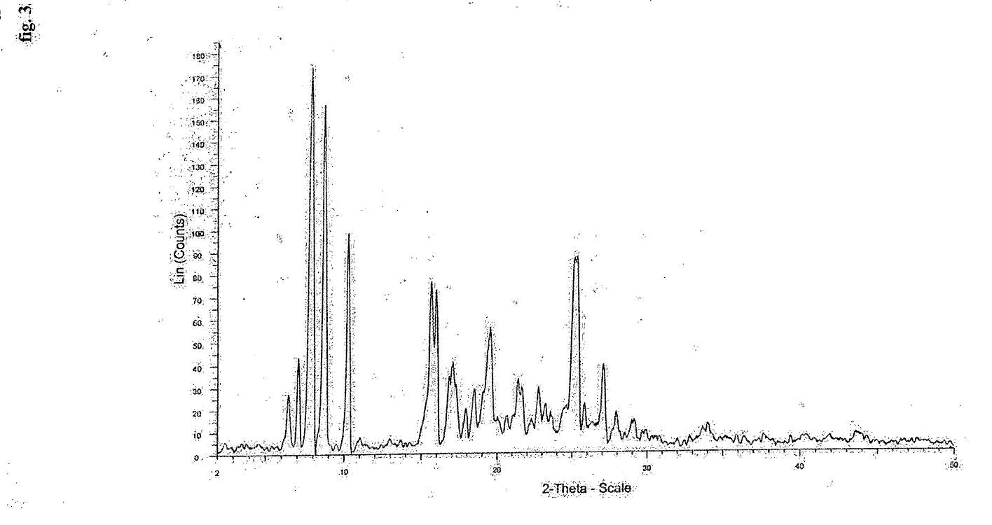

9. A crystalline tegaserod maleate Form III as defined in claim 8, further characterized by an x-ray powder diffraction pattern as shown in FIG. 3.

10. A process for preparing tegaserod maleate Form III as defined in claim 8, which comprises:
a) mixing maleic acid and a solution of tegaserod free base in methanol; and
b) collecting the solid separated by filtration.

11. A process for preparing tegaserod maleate Form III as defined in claim 8, which comprises;
a) dissolving tegaserod maleate in methanol;
b) maintaining for about 30 minutes at about 20° C. to 25° C. to produce a solid; and
c) collecting the solid by filtration.

12. A crystalline tegaserod maleate Form IV, characterized by an x-ray powder diffraction pattern having peaks expressed as 2θ at about 6.9, 8.0, 10.3, 16.5, 19.6, 20.4, 20.9, 22.0, 23.2, 25.4, 28.0 and 28.7 degrees.

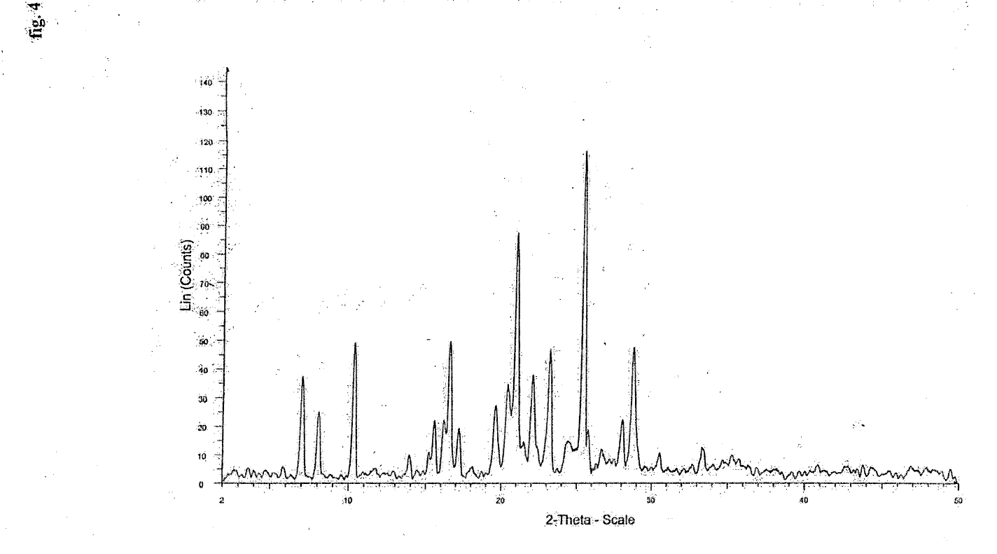

13. A crystalline tegaserod maleate Form IV as defined in claim 12, further characterized by an x-ray powder diffraction pattern as shown in FIG. 4.

14. A process for preparation of tegaserod maleate Form IV as defined in claim 12, which comprises:
a) mixing maleic acid and a solution of tegaserod free base in methanol; and
b) precipitating tegaserod maleate Form IV by mixing with methylene dichloride or isopropyl alcohol.

15. A pharmaceutical composition comprising a crystalline form of tegaserod maleate and a pharmaceutically acceptable carrier.

16. A pharmaceutical composition as defined in claim 15, wherein the crystalline form is the tegaserod maleate Form I of claim 1.

17. A pharmaceutical composition as defined in claim 15, wherein the crystalline form is the tegaserod maleate Form II of claim 5.

18. A pharmaceutical composition as defined in claim 15, wherein the crystalline form is the tegaserod maleate Form III of claim 8.

19. A pharmaceutical composition as defined in claim 15, wherein the crystalline form is the tegaserod maleate Form IV of claim 12.
